(19) United States
(12) Patent Application Publication
(10) Pub. No.: US 2005/0158256 A1
(43) Pub. Date: Jul. 21, 2005

(54) Cosmetics tester and method of manufacturing such a tester

(75) Inventor: Thomas Berger, Saint Sulpice (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/001,009

(22) Filed: Dec. 2, 2004

Related U.S. Application Data
(60) Provisional application No. 60/528,498, filed on Dec. 11, 2003.

(30) Foreign Application Priority Data
Dec. 2, 2003 (FR) 03 50956

(51) Int. Cl. A61K 7/04; A61K 7/021; A61K 9/70

(52) U.S. Cl. 424/61; 424/443

(57) ABSTRACT

A cosmetics tester including a transparent support medium in the form of a sheet. The support medium is partly covered by a motif. Preferably at least two visually different motifs are provided which are formed from at least one cosmetic composition, for example, a nail varnish, with the compositions being permanently attached to the support medium. The invention also relates to a method of manufacturing a cosmetics tester wherein at least one cosmetic composition is screen-printed onto a transparent support medium so as to obtain a motif capable of being permanently attached to the support medium.

### CROSS REFERENCE TO RELATED APPLICATIONS:

This document claims priority to French Application Number 03 50956, filed Dec. 2, 2003 and U.S. Provisional Application No. 60/528,498, filed Dec. 11, 2003, the entire contents of which are hereby incorporated by reference.

### FIELD OF THE INVENTION

The invention concerns a cosmetics tester capable of viewing a cosmetic composition without applying it, and a method of manufacturing such a tester.

### BACKGROUND OF THE INVENTION

### DISCUSSION OF BACKGROUND

A composition is said to be cosmetic when it contains a physiologically acceptable medium, i.e., it can be applied to the skin, face, lips and other external parts of the human body such as the nails, eyelashes, eyebrows or hair.

When buying a cosmetic product, the purchaser generally wishes to be able to view the cosmetic composition before use. Certain cosmetics are packaged in transparent containers which enable the color of the cosmetic composition to be seen. This notably applies to nail varnishes which are generally packaged in transparent bottles. However, the visual appearance of the composition in a container holding a large volume of product does not always match the visual appearance of a thin layer of the composition on the skin or nails. For this reason, in the case of nail varnishes for example, certain stores provide test cards on which coats of nail varnish are applied, which provides a better idea of how a layer of the nail varnish composition will look on a nail.

When a cosmetic is purchased by mail order, the product must be selected on the basis of the color printed in a catalog. However, it is very difficult to reproduce the colors of a layer of a cosmetic composition on the page of a catalog. It is also very difficult to reproduce on the page of a catalog the effects created by certain compositions such as, in the case of nail varnish for example, a mohair effect, an interference effect (the visual appearance of the composition varies in relation to angle from which it is viewed), a shimmer effect, etc.

In the same manner, documents FR 1 110 340 and FR 2 227 805 describe transparent strips colored to represent different parts of the face or hands, notably the lips, eyebrows or nails in various shades. Here again, it is very difficult to reproduce the color of a layer of a cosmetic composition on such a strip.

Product testers also exist that include a small quantity of product designed to be sampled by the user and applied in order to give an idea of the appearance of the product before making a purchase. For example, U.S. Pat. Nos. 4,751,934; 4,848,378; 5,192,386; 5,799,675; and EP 1,266,589 describe such testers.

### SUMMARY OF THE INVENTION

One of the objects of the invention is to provide a tester which allows the finished appearance of a layer of a cosmetic composition to be viewed without having to apply it.

It is a particular object of the invention to simulate the application of a layer of a cosmetic composition onto an area of the skin or onto a nail.

It is a further object of the invention to provide such a tester that is of compact size and relatively lightweight, while being sufficiently rigid to be capable of being mailed and transported without risk of damage.

According to the invention, these aims can be achieved by providing a cosmetics tester including a transparent support medium in the form of a sheet. In accordance with a preferred example, the support medium is partly covered by at least two visually different motifs formed from at least one cosmetic composition, notably a nail varnish, with the two compositions permanently attached to the support medium.

For the purposes of the present invention, the expression “compositions attached permanently to the support medium” means compositions that are not transferable from the support medium to the skin or nails for normal use. Unlike testers containing a quantity of product to be applied as previously described, such compositions cannot be taken up by a finger to be applied to the skin or nails. The compositions can include for example nail varnish, transfer-resistant lip color, transfer-resistant foundation, etc. Of course such compositions can be removed, at least partially, from the support medium by means of a product such as a solvent-based remover in the case of nail varnish or by means of a make-up remover in the case of other cosmetic products.

Using the cosmetic composition itself, which is applied to a transparent support medium, a tester is obtained that can more optimally (preferably perfectly) reproduce the effect obtainable by applying the composition, for example, to the skin or nails. In effect, because the support medium is clear, there is no risk of it impairing the visual appearance of the composition itself.

Various motifs can be visually different, and the same tester can be used to view different cosmetic compositions, for example the full assortment of shades in a cosmetic product range or line.

By way of example, the cosmetic composition can contain an organic or aqueous solvent medium and at least a filmformer polymer, with the motif being formed by a film resulting from the evaporation of the solvent phase of the composition, after deposit of the aforementioned cosmetic composition on the support.

The motifs can be formed from two different cosmetic compositions, distinguished notably by their color and/or their texture.

Alternately, or in addition, the motifs can differ in terms of their shape. They can notably have a shape selected from the shape of a nail, a lip, an eyelid, a cheek, depending on the cosmetic composition constituting the motif. This makes it possible to obtain an even better match with the effect obtained when the composition is applied to the skin or the nails. It is also easy to simulate the application of a layer of the composition by superimposing the motif over the area of the body to which the product is to be applied without actually having to apply a layer of the composition.

The transparent support medium can be made of a thermoplastic material.

The sheet can have a thickness of between 100 and 300 μm and preferably between 100 and 200 μm. Such a tester is therefore of compact dimensions and can for example be easily slid inside a catalog. In addition, it is sufficiently lightweight and rigid to be capable of being mailed or transported without risk of damage.

The cosmetic compositions can be deposited on the carrier sheet by screen-printing.

The invention also relates to a method of manufacturing a cosmetics tester wherein at least one cosmetic composition is provided, for example screen-printed, onto a transparent support medium so as to obtain a motif capable of being permanently attached to the support medium.

The screen-printing method makes it possible to deposit a cosmetic composition to a greater or lesser thickness by varying the mesh size of the screen used for the printing process.

Several different cosmetic compositions can be printed onto the support medium, and the support medium can be, for example, a sheet of thermoplastic material.

### BRIEF DESCRIPTION OF THE DRAWINGS

The invention will become further apparent from the following detailed description, particularly when considered in conjunction with the drawings in which:

FIG. 1 illustrates a perspective view of a tester according to the invention;

FIG. 2 shows a cross-sectional view of the tester depicted in FIG. 1; and

FIG. 3 illustrates a perspective view of the tester according to the invention during use.

### DETAILED DESCRIPTION OF THE PREFERRED EMBODIMENTS

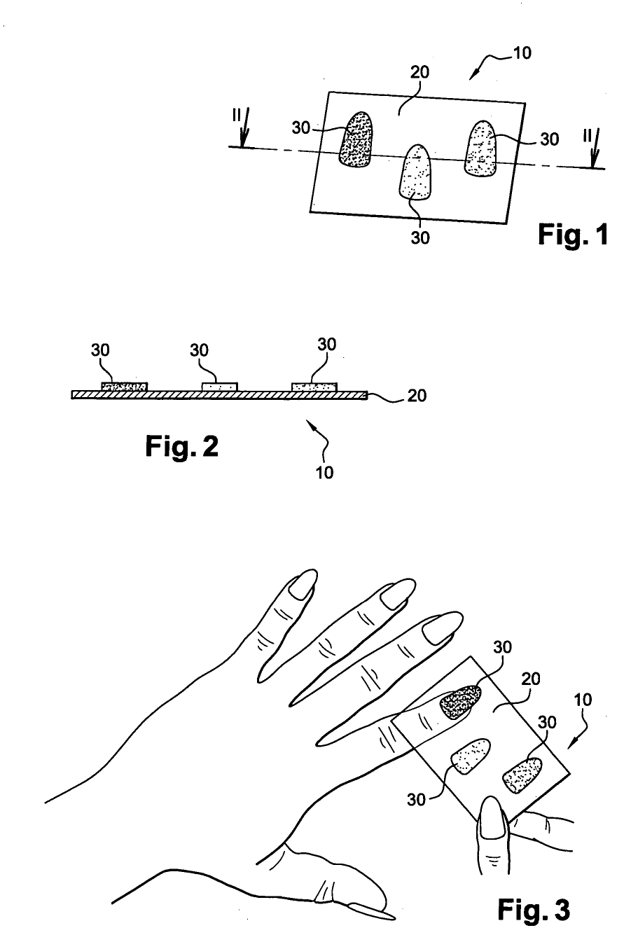

In the example embodiment illustrated in FIGS. 1 to 3, the tester 10 is designed to view a nail varnish composition.

The tester 10 is formed by a transparent support medium 20 in the form of a sheet, with the sheet being partially covered by a nail varnish composition.

In the example shown, the support 20 is a sheet having, by way of example, a thickness of approximately 130 μm, a width of 5 cm, and a length of 10 cm. The support medium is therefore relatively light in weight and of compact dimensions.

The support medium is made of polystyrene for example, notably clear polystyrene marketed by the company Sidaplast under the designation PF 110 Cristal Clear. Such a material is very clear and provides good adhesion of the nail varnish composition, without deformation of the support medium.

In the illustrated example, the support medium 20 includes three motifs 30 in the shape of finger nails each formed by a layer of a nail varnish composition. Each motif is made from a different nail varnish composition. Preferably, each motif has a different visual appearance, in particular, a different color and/or shade, and/or a different effect, for example a shimmer effect, an interference effect, a mohair effect, etc.

It is evident that the support medium could include even more motifs to enable the user to view several different nail varnish compositions, for example all of the available shades in the same range of nail varnish.

The layers of the nail varnish compositions have a thickness of approximately 30 μm. Such a layer therefore optimally simulates a layer of the composition applied to a nail.

To use such a tester, the user grasps the support medium 20 and superimposes one of the motifs 30 over her finger nail, as can be seen in FIG. 3. She is thus readily able to simulate the application of a layer of the nail varnish composition onto her nail without actually having to apply it. The support medium is in effect sufficiently rigid to be able to remain substantially flat while being held in one hand by only one of its corners and being superimposed over the other hand.

To produce the tester 10, a sheet of polystyrene is made in a relatively large format. One or more layers of nail varnish composition are then screen-printed onto the sheet so as to form one or more motifs 30 in the shape of nails.

For example, a flat silk-screen process can be used with a mobile screen which can include several nail-shaped motifs, and a fixed squeegee. The polystyrene sheet is brought into contact with the screen against which it is held, opposite the squeegee, by means of a counter roller. After depositing the nail varnish composition(s) on the screen, the sheet is moved in the same direction as that of the screen and the squeegee causes the composition to pass through the screen mesh forming the motifs, thereby transferring it to the sheet. When making a support medium covered with several different nail varnish compositions, they can be printed in the same screen-printing pass or in several different passes.

The sheet thus printed is then dried, for example, with ambient air in a drying tunnel so as to extract the solvents from the nail varnish compositions.

After a drying time of approximately 30 seconds, the sheets thus obtained are stacked together and trimmed to obtain the format of the support medium 20 including, as illustrated in the example in FIG. 1, three motifs 30.

The tester 10 thus obtained can be associated with a cosmetics mail order catalog, for example, with the tester slid inside the catalog.

The tester 10 can also be used in stores, at points of sale for cosmetic products, and made available to customers. This allows salespersons to avoid having to apply nail varnish to customers' nails and subsequently removing it using a solvent remover. The tester 10 can also be reused several times for several customers.

In an example not shown, the tester can be formed by a transparent sheet partly covered by a transfer-resistant lip color composition. In this case the motif is lip-shaped.

Obviously, numerous modifications and variations of the present invention are possible in light of the above teachings. It is therefore to be understood that, within the scope of the appended claims, the invention may be practiced otherwise than as specifically described herein.

1. A cosmetic tester including:
a transparent support medium in the form of a sheet;
at least two visually different motifs which partially cover the sheet, wherein the at least two visually different motifs are formed from at least one cosmetic composition, and wherein the at least two visually different motifs are permanently attached to the support medium.

2. A tester according to claim 1, wherein the at least one composition comprises a nail varnish.

3. A tester according to claim 2, wherein the cosmetic composition contains an organic or aqueous solvent medium and at least a filmformer polymer, and wherein each motif is formed by a film resulting from evaporation of the solvent of the composition after deposit of the cosmetic composition on the support.

4. A tester according to claim 3, wherein the at least two motifs are formed from two different cosmetic compositions distinguished by their color.

5. A tester according to claim 3, wherein the at least two motifs are formed from two different cosmetic compositions distinguished by their texture.

6. A tester according to claim 1, wherein the at least two motifs are formed from two different cosmetic compositions distinguished by their color.

7. A tester according to claim 1, wherein the at least two motifs are formed from two different cosmetic compositions distinguished by their texture.

8. A tester according to claim 1, wherein the motifs having different shapes.

9. A tester according to claim 1, wherein the motifs have a shape selected from the shape of a nail, a lip, an eyelid, or a cheek.

10. A tester according to claim 1, wherein the transparent support medium is made of a thermoplastic material.

11. A tester according to claim 10, wherein the sheet has a thickness of between 100 and 300 μm.

12. A tester according to claim 10, wherein the sheet has a thickness of from 100 to 200 μm.

13. A tester according to claim 10, wherein the at least one cosmetic composition is applied by screen-printing.

14. A tester according to claim 1, wherein the at least one cosmetic composition is applied by screen-printing.

15. A tester according to claim 1, wherein the transparent support medium is formed with a polystyrene material.

16. A tester according to claim 15, wherein the at least one cosmetic composition includes a nail varnish.

17. A tester according to claim 15, wherein the at least two motifs are screen-printed on the polystyrene.

18. A method of manufacturing a cosmetics tester comprising:
providing a transparent support medium;
screen-printing at least one cosmetic composition onto the transparent support medium so as to obtain a motif capable of being permanently attached to the support medium.

19. A method according to claim 18, further including screen-printing a plurality of different cosmetic compositions on the support medium.

20. A method according to claim 19, wherein the support medium is a sheet of thermoplastic material.

21. A method according to claim 18, wherein the support medium is a sheet of thermoplastic material.

22. A method according to claim 18, wherein the support medium is formed with a polystyrene material.

23. A method according to claim 22, wherein the at least one cosmetic composition includes a nail varnish.

24. A method according to claim 18, wherein the at least one cosmetic composition includes a nail varnish.

25. A method according to claim 18, wherein at least two motifs having different textures are screen-printed on the support medium.

26. A method according to claim 18, wherein at least two motifs having different colors are screen-printed on the support medium.

27. A cosmetic tester comprising:
a transparent support medium in the form of a sheet;
at least one motif which partially covers the sheet and which is screen-printed on the sheet;
wherein the at least one motif is formed from at least one cosmetic composition and is permanently attached to the sheet.
